(19) United States
(12) Patent Application Publication
(10) Pub. No.: US 2023/0046095 A1
(43) Pub. Date: Feb. 16, 2023

(54) DEVICE FOR DETERMINING A PHYSIOLOGICAL CONDITION OF A PERSON

(71) Applicant: XOTOX TOOLS AG, CH-8700 KUSNACHT (CH)

(72) Inventors: Philipp BACHMANN, Kusnacht (CH); Rolf FRITZSCHE, Hinwil (CH)

(73) Assignee: XOTOX TOOLS AG, CH-8700 KUSNACHT (CH)

(21) Appl. No.: 17/965,962

(22) Filed: Oct. 14, 2022

Related U.S. Application Data
(63) Continuation of application No. 16/180,602, filed on Nov. 5, 2018.
Application No. 16/180,602 is a continuation-in-part of application No. 14/435,586, filed on Apr. 14, 2015, now abandoned.

(30) Foreign Application Priority Data
Oct. 16, 2012 (EP) 12188701.2

(51) Int. Cl. A61B 5/16; A61B 5/024; A61B 5/0245; A61B 5/316; A61B 5/332

(52) U.S. Cl. CPC A61B 5/165; A61B 5/02405; A61B 5/02438; A61B 5/02455; A61B 5/316; A61B 5/332; A61B 2503/20; A61B 5/024; A61B 5/746

(57) ABSTRACT

A device for determining the physiological condition of a person in a simple manner, which device measures the pulse rate and based on that additionally determines the heart rate variability. In addition, at least one parameter should be used for the history of one of the two above-mentioned values. The deviation of the pulse rate and the heart rate variability from a normal variable is preferably integrated and used as an additional indicator. The device preferably includes a wearable electrocardiography device.

### CROSS REFERENCE TO RELATED APPLICATION

This application is a continuation of co-pending U.S. patent application Ser. No. 16/1480,602, filed on 5 Nov. 2018 which is a continuation-in-part of U.S. patent application Ser. No. 14/435,586, filed on Apr. 14, 2015, which claims priority from PCT application No. PCT/EP2013/069647, filed Sep. 20, 2013, which claims priority from European application No. EP 12188701.2 filed on Oct. 16, 2012, the disclosures of which are incorporated herein by reference.

### BACKGROUND OF THE INVENTION

### Field of the Invention

The invention relates to a device and a method for detecting and reporting of a stress condition of a person.

### Discussion of Related Art

Using the pulse rate or the heart rate variability for the detection of stress conditions has been known for a long time. Thereby, the interval between two heart beats is defined, in the sense of the present invention, as the time between the onsets of two contractions of the cardiac chambers. This onset of the chamber contraction shows up in the electrocardiogram (ECG) as the R wave. The distance between two R waves is usually denoted as RR interval. After averaging over a defined number of RR intervals, the heart rate can be determined by calculation. The individual values of the RR intervals vary around the mean value thus obtained. Thereby the variations can change from beat to beat. The variation is usually denoted as heart rate variability (HRV). In principle, the heart rate can also be determined by a pressure measurement carried out on an artery.

Physiologically, the heart rate variability (HRV) is related to the ability of the human organism to adapt the rate of the cardiac rhythm. Variations of the heart rate, i.e., variations of the temporal interval between two heart beats, can occur in a resting state, in which case they are mostly spontaneous, but also upon specific variations of the surrounding conditions, e.g., under stress. A healthy organism continuously adapts the heartbeat rate to the current conditions via physiological regulation pathways of the vegetative nervous system. Therefore, physical or psychological stress usually results in an increase of the heart frequency which ordinarily decreases again upon relief and relaxation. Thereby, a good adaptability to stress results in a higher variability of the heart rate. Under chronic stress burden, the adaptability is reduced. In this respect, it is known that the heart rate variability taken by itself already provides a certain, albeit still very unreliable, indicator for the current stress burden and the ability of a person to cope with stress.

Several methods for determining the stress condition of a person have been proposed in the prior art, including the proposal to use further measurement parameters in addition to the pulse rate. Thus, DE 103 19 361 A1 proposes to use the pulse wave latency in addition to the heart rate variability.

Regarding the analysis of the heart rate variability, reference is made to DE 100 06 154 A1, DE 10 2006 039 957 A1, and also to DE 10 2008 030 956 A1 and EP 1 156 851 B1, in which the person skilled in the art can find various determination methods.

From EP 2 316 333 A1 there is known a device and a method in which a state quantity is calculated which is a function of the current heart rate and of the current heart rate variability, preferably a linear combination of the current heart rate P and of the current heart rate variability. Moreover, in the mentioned publication, it is proposed to provide the state function with at least one correcting value that includes the history of the person within at least the past 0.5 hours.

This approach appears quite suitable here. However, there results a further optimization problem which can be described by the fact that, on the one hand, particularly for comparing different persons, it is necessary to introduce normalizations, but that on the other hand it is also necessary to take into account individual differences.

### SUMMARY OF THE INVENTION

The object of the invention is to provide a device and a corresponding method for detecting and reporting of a stress condition of a person with an increased reliability as compared to the state of the art, and in which, on the one hand, particularly for comparing different persons, normalizations can be introduced, but in which, on the other hand, individual differences are also taken into account.

At the outset, the features of the invention have the result that, to a first approximation, namely in the first processing window, i.e., in a first-time interval T1 or across a predetermined number of pulse beats, a first approximation result is obtained that can already be processed. For this purpose, one may consider, in particular, age-related values that provide, preferably, for the respective age groups, typical tabulated values of the minimum and the maximum values of the pulse rate and the HRV value. The subsequent processing windows then can, according to experimental studies, provide results that are improved, particularly if the person has characteristics that deviate from the tabulated values of the minimum and the maximum values of the pulse rate and the HRV value, regardless of whether this is due to reasons of e.g., a particularly good or particularly bad training condition or whether it is also due to high or low stress values in the near or medium term history. According to the present invention, pulse rate and HRV shall be measured by means of a wearable electrocardiography device. In the present context, the term “wearable electrocardiography device” shall be understood as a device that can be worn by a person for an extended period of time, preferably continuously for 24 hours a day and 7 days a week, without need for substantial interruption of regular physical and mental activities such as work, leisure and sports. The term “electrocardiography” refers to a method of acquiring electrophysiological activity of the wearer's heart. Electrical signals obtained as a function of time allow for precise determination of instant heartbeat frequency (expressed as a frequency, typically a number of heart beats per minute). The often-used terms “pulse rate” or “pulse frequency” are due to the fact that many measurements are taken at the pulse of a person. For the purpose of the present invention, these are equivalent terms to denote the heartbeat frequency. Heart rate variability (expressed as time derivative of a frequency). The use of an electrocardiography device is essential in order to achieve the accuracy required by the present invention, which should be in the range of less than 10% of missed heartbeats over a 24-hour period. Such accuracy is typically not achieved by simpler devices often implemented in wearable devices. Heart rate variability is obtained by statistical evaluation of heartbeat frequency over a certain sampling period.

According to the method for detecting and reporting of a stress condition of a person according to the present invention, the following steps are carried out:
continuously acquiring data of a current pulse frequency P and of a current heart rate variability HRV of said person by means of a wearable electrocardiography device,
continuously processing the data of the current pulse frequency P and of the current heart rate variability HRV,
determining a stress index and comparing the stress index with an alarm value indicative of an occurrence of said stress condition.

Detecting the occurrence of a stress condition has numerous practical applications. These include, e.g., inducing exposed workforce such as fireman or protective force to take a recovery period when their stress level reaches what can be termed an alarm level. Similarly, a stress condition may be detected in athletes, but also in any other persons subject to a physically and/or mentally demanding situation.

Said determining of the stress index comprises:

within a first time interval T1, or across a predetermined number of pulse beats, a first value SI1 for the stress index is determined by adding a value SIP, which is obtained from a normalized average value Pd1 of the pulse frequency in said first time interval T1 or across said predetermined number of pulse beats, plus a value SIHRV, which is obtained from a normalized average value HRVd1 of the heart rate variability HRV within said first time interval T1 or across said predetermined number of pulse beats according to:

SI1=c*SIP+d*SIHRV

wherein normalization is carried out by means of tabulated values Pₘₐₓ, Pₘᵢₙ, HRVₘₐₓ and HRVₘᵢₙ obtained from age dependent minimum and maximum pulse frequency values and HRV values, and, furthermore, the maximum and minimum values of the measured pulse frequency values and HRV values within the time interval T1 or across the predetermined number of pulse beats are determined,

wherein T1 lies between 100 s and 1000 s, or the predetermined number of pulse beats lies between 50 and 500,

in at least one further time interval Tx (x=2 . . . n), or across a further predetermined number of pulse beats, determining a further value SIx for the stress index by adding a value SIP which is obtained from a normalized average value Pd1 of the pulse frequency in said further time interval Tx or across said further predetermined number of pulse beats, plus a value SIHRV, which is obtained from a normalized average value HRVd1 of the heart rate variability HRV within said further time interval Tx or across said further predetermined number of pulse beats according to:

SI_{x=c}*SI_{P+d}*SIHRV

wherein said further time interval Tx has the same length as said first time interval T1 and wherein said further predetermined number of pulse beats is the same as that predetermined number of pulse beats,

wherein normalization is carried out by means of values Pmax, Pₘᵢₙ, HRVₘₐₓ and HRVₘᵢₙ, wherein Pmax and HRVₘₐₓ are selected from the larger value between Pmax and HRVₘₐₓ determined in the previous time interval Tx-1 or across the predetermined number of pulse beats, respectively, and the values of Pmax and HRVₘₐₓ used in the previous time interval Tx-1 or across the predetermined number of pulse beats, respectively, and wherein Pₘᵢₙ and HRVₘᵢₙ are selected from the smaller value between Pₘᵢₙ and HRVₘᵢₙ determined in the previous time interval Tx-1 or across the predetermined number of pulse beats, respectively, and the values of Pₘᵢₙ and HRVₘᵢₙ used in the previous time interval Tx-1 or across the predetermined number of pulse beats, respectively,

thereby obtaining said stress index as being equal to said further value SIx.

Within the time interval, or for a predetermined number of pulse beats, the data of the current pulse frequency P and of the current heart rate variability HRV are continuously acquired and processed. Thereby, a first value for the stress index is separately calculated for the pulse rate and for the heart rate variability, and subsequently the two values are added, using weighting factors. In this step the values of the pulse rate and of the heart rate variability are normalized with respect to tabulated extremal values Pmax, Pₘᵢₙ, HRVₘₐₓ and HRVₘᵢₙ, which extremal values have preferably been sorted according to age. In this manner a first, potentially already useful, value for the stress index is determined.

Subsequently, however, one determines for said time interval, or for said predetermined number of pulse beats, whether the individual extremal values Pₘₐₓ, Pₘᵢₙ, HRVₘₐₓ and HRVₘᵢₙ are different from the previously adopted values. For the subsequent time interval or for the subsequent measurement of predetermined pulse beats, for which neither the length of the time interval nor the number of the pulse beats necessarily needs to be identical to that of the first measurement, and which can also be significantly later, i.e. after a temporal delay of up to several hours, either the earlier extremal values or the newly determined extremal values are used, depending on which values are more extremal. The calculation of the new values for the stress index then occurs basically in an analogous manner as the above described first measurement, i.e., a value of the stress index is calculated by continuously and separately determining the stress index values for the pulse rate and for the heart rate variability and subsequently adding the two values with weighting factors. The time interval lies typically in the range of 100 s to 1000 s, preferably of 300 s to 500 s, but it can also be about one order of magnitude (factor 10) smaller or larger. The predetermined number of pulse beats lies between 50 and 500, preferably 100, but it can be larger, in particular, by about one to two orders of magnitude. It should be emphasized that the subsequent measurements may temporally overlap, in which case at the onset of the new series of measurements clearly only those extremal values can be used that have previously occurred. This “moving-window” method can be appropriate if massive computing power is available, and a very rapid result shall be achieved.

Advantageously, the method can be carried out if the normalizations are each carried out by means of a normalization value at the first, table-related

P_{z}=Pₘᵢₙ+a*(Pₘₐₓ−Pₘᵢₙ)

HRV_{z}=HRVₘᵢₙ+b*(HRVₘₐₓ−HRVₘᵢₙ)

and the calculations of the summands of the stress value SI are each carried out according to

SIP=(Pd1−P_{z})/(Pₘₐₓ−P_{z}) if Pd1>P_{z}

SIP=(Pd1−P_{z})/(P_{z}−Pₘᵢₙ) if Pd1<P_{z}

SIHRV=−(HRV_{d1}−HRV_{z})/(HRVₘₐₓ−HRV_{z}) if HRV_{d1}>HRV_{z}

SIHRV=−(HRV_{d1}−HRV_{z})/(HRV_{z}−HRVₘᵢₙ) if HRV_{d1}<HRV_{z}.

The further windows are then advantageously calculated according to

SIP=(Pdₓ−P_{z})/(Pₘₐₓ−P_{z}) if Pdₓ>P_{z}

SIP=(Pdₓ−P_{z})/(P_{z}−Pₘᵢₙ) if Pdₓ<P_{z}

SIHRV=−(HRV_{dx}−HRV_{z})/(HRVₘₐₓ−HRV_{z}) if HRV_{dx}>HRV_{z}

SIHRV=−(HRV_{dx}−HRV_{z})/(HRV_{z}−HRVₘᵢₙ) if HRV_{dx}<HRV_{z}.

It has been proven advantageous if the values a for the processing of the pulse rate are selected in the range between 0.2 and 0.3, advantageously as 0.25 and the values b for the processing of the heart rate variability between 0.33 to 0.66, advantageously as 0.5.

Depending on the application the method can be calibrated in the sense that the weighting of the partial value of the stress index, which is determined from the pulse rate, and of the partial value of the stress index, which is determined from the heart rate variability, are optimized by trials. If there are no reasons to do so, the values c and d will be selected as 1.

It is particularly advantageous if the respectively determined stress values are not only output directly, which of course is not ruled out, but rather the current stress index SI is fed to a filter, typically to a digital low-pass filter, after the time interval Tx or after the predetermined number of pulse beats, in order to average over individual outliers, e.g. SI=f*SIx+(1−f)*SIx−1, with f between 0.05 and 0.5, advantageously of 0.1.

According to a second aspect of the present invention there is proposed a device that is suitable for carrying out the method of the present invention, optionally including the above-mentioned advantageous embodiments.

Such a device for detecting and reporting of a stress condition of a person typically comprises an acquisition device for continuously acquiring data of the current pulse frequency and of the current heart rate variability, a processing device for continuously processing the data of the current pulse frequency and of the current heart rate variability, and a comparator device for comparing the current state function of the person thus obtained with an alert criterion.

The aforementioned elements as well as those claimed and described in the following exemplary embodiments, to be used according to the invention, are not subject to any particular conditions by way of exclusion in terms of their size, shape, use of material and technical design, with the result that the selection criteria known in the respective field of application can be used without restrictions. For carrying out the method of the present invention, the calculation of the heart rate variability can be carried out by means of conventional methods (e.g., in the time domain: RMSSD, RRinter, SDNN, or in the frequency domain LFtot/HFtot), wherein the method RMSSD (“Root mean square of successive differences”) is suitable for the method and device mentioned here. It should be pointed out that the method of the present invention is not intended for determining the health status or pathological status of the person, whereas the device of the present invention is not restricted in this regard.

In order to arrive at the above-mentioned stress index components, both independent claims use a step of normalizing certain values obtained from the primary data, i.e., from the pulse rate and heart rate variability derived from measurement. Such normalization leads to the following instantaneous values:
normalized average value of the pulse frequency
normalized average value of the heart rate variability.

Normalization starts out in both cases by scaling with respect to a normalizing range, with the latter being defined by a difference of maximum and minimum values, i.e., Pₘₐₓ−Pₘᵢₙ, and HRVₘₐₓ−HRVₘᵢₙ, respectively.

The external reference values Pmax, Pₘᵢₙ, HRVₘₐₓ and HRVₘᵢₙ are age-dependent reference values, i.e., as maximum and minimum values derived from an age-matched population.

While determination of an instantaneous stress index starts out in the manner described above, i.e., by using external reference values, it then goes on to incorporate an individual, not externally given, normalization criterion which takes into account the characteristics of the person of interest. The switch from a frozen normalization anchored to external reference values to a dynamical individual-specific normalization occurs in the step where the parameters Pmax, Pₘᵢₙ, HRVₘₐₓ and HRVₘᵢₙ are selected as the respective extremal values in the preceding data acquisition history.

The above explained dynamic normalization procedure comprising a step of processing high-precision individual data is equivalent to the processing of a potentially noisy signal in a dynamically changing framework, which in the present invention consists of an individual's continuously changing situation. The latter could be understood as a momentary “stress load”. Accordingly, the results obtained with the present invention, i.e., a momentary stress index which will be either above or below an appropriately chosen alarm criterion, are found to be substantially improved as compared to a simple approach in which a measured pulse rate and/or HRV is simply compared with predefined external threshold values.

### BRIEF DESCRIPTION OF SEVERAL VIEWS OF THE DRAWINGS

The above mentioned and other features and objects of this invention and the manner of achieving them will become more apparent and this invention will be better understood by reference to the following description of various embodiments of the invention taken in conjunction with the accompanying drawings,

FIG. 1 shows a block diagram representation of a device for detecting and reporting of a physiological condition of a person; and

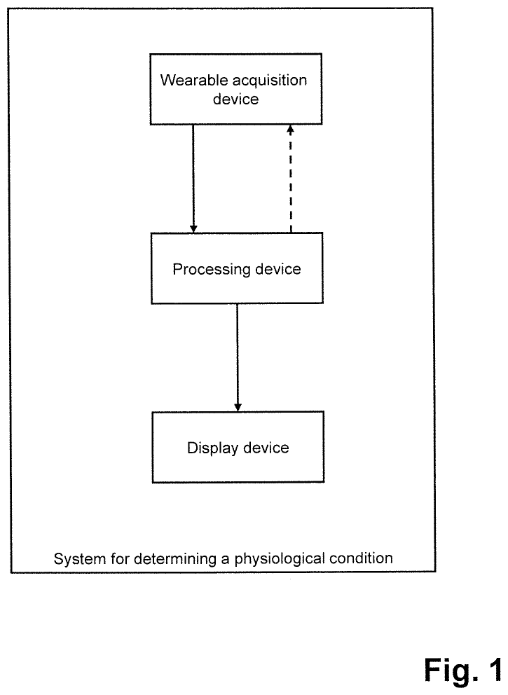

FIG. 2 shows a block diagram representation including a representation of the steps for determining a physiological condition.

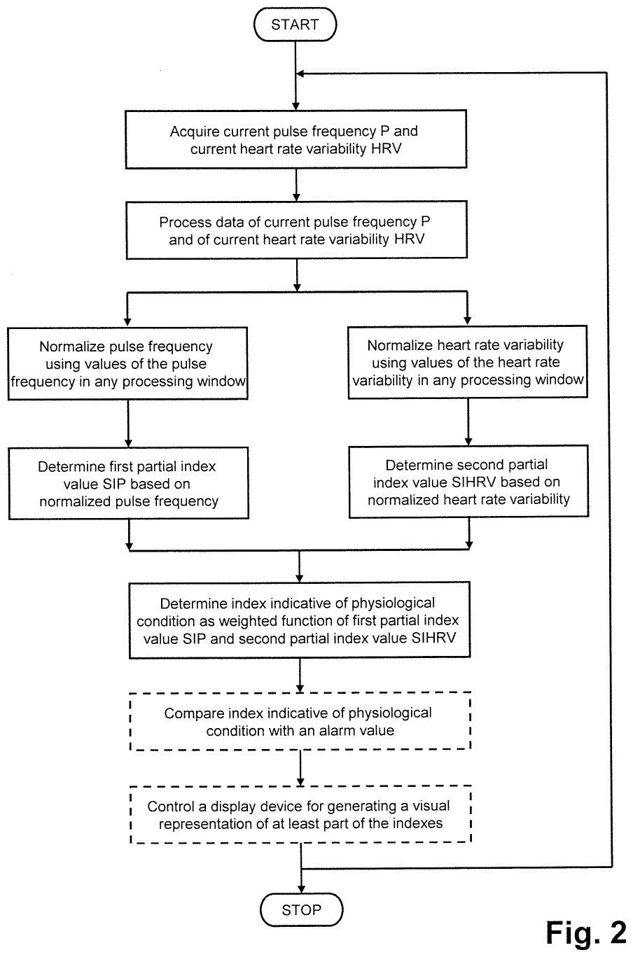

### DETAILED DESCRIPTION OF THE INVENTION

The device of the present invention comprises, according to a preferred exemplary embodiment of the invention, a measuring device for detecting the pulse rate and the values that are necessary for calculating heart rate variability. In the present case this is a pulse measuring sensor, but alternatively it can also be an electrical sensor for measuring electrical cardio graphic measurement values, as well as a display device. Moreover, the device comprises an interface for the input of person-related parameters, which are particularly needed for determining the history to be used according to the invention. A key component of the device is a computing device that controls the necessary acquisition of the measurement data, processes the measurement data in the digital form needed, executes the data processing and controls the display.

In the present exemplary embodiment the heart rate variability HRV is determined by means of the RMSSD method (“Root mean square of successive differences”), but also by other methods, such as e.g. the method “SIR” based on standard deviations, the method pRR50, in which the number of consecutive RR intervals that are larger than 50 ms is determined and the value thus obtained is divided by the total number of consecutive RR intervals, or frequency-oriented methods such as, for example, the calculation via the quotient LFtot/HFtot of the low-frequency frequency components divided by the higher-frequency frequency components. The HRV value obtained by means of RMSSD is calculated as the square root of the sum of the squared differences between neighboring RR intervals. In this context it should be noted that for the selection of the calculation method one may use, on the one hand, pertinent recent findings of the respective technical field and of the respective application range of the method according to the present invention or of the device according to the present invention, but on the other hand it is conceivable to simply take into account practical aspects of the respective selection. In the case where the HRV values are determined by means of the RMSSD method, in the present exemplary embodiment 0 is used as tabulated value for HRVₘᵢₙ for all ages. The other minimum values used in the exemplary embodiment shown here, which refer to the pulse and to the HRV, are selected according to the following table:

Age dependent resting heart rate values

| | |
|---|---|
| Youths: 14 . . . 18 | Resting heart rate: 85 beats/minute |
| Adults: 19 . . . 65 | Resting heart rate: 70 beats/minute |
| Seniors: 65+ | Resting heart rate: 90 beats/minute |

Age dependent HRVₘₐₓ values (RMSSD)

| | |
|---|---|
| 15 . . . 20 | 47 ms |
| 21 . . . 30 | 46 ms |
| 31 . . . 40 | 40 ms |
| 41 . . . 50 | 35 ms |
| 51 . . . 60 | 30 ms |
| 61 . . . 70 | 24 ms |

according to Angelink et al: Innovationstagung FH Rapperswil 4.5.2011

In this context it should be noted that, without departing from the sense of the method of the present invention, rather different parameters of the subjects such as e.g., the gender etc. can be incorporated into the table.

According to the exemplary embodiment the normalizations are each carried out by means of a normalization value

P_{z}−Pₘᵢₙ+a*(Pmax−Pₘᵢₙ)

HRV_{z}=HRVₘᵢₙ+b*(HRVₘₐₓ−HRVₘᵢₙ)

and the calculation of the summands of the stress value SI is each carried out according to

SI_{P}−(P_{d1}−P_{z})/(Pₘₐₓ−P_{z}) if P_{d1}>P_{z}

SI_{P}=(P_{d1}−P_{z})/(P_{z}−Pₘᵢₙ) if P_{d1}<P_{z}

SI_{HRV}=−(HRV_{d1}−HRV_{z})/(HRVₘₐₓ−HRV_{z}) if HRV_{d1}>HRV_{z}

SI_{HRV}=−(HRV_{d1}−HRV_{z})/(HRV_{z}−HRVₘᵢₙ) if HRV_{d1}<HRV_{z}

and

SI_{P}=(P_{dx}−P_{z})/(Pₘₐₓ−P_{z}) if P_{dx}>P_{z}

SI_{P}=(P_{dx}−P_{z})/(P_{z}−Pₘᵢₙ) if P_{dx}<P_{z}

SI_{HRV}=−(HRV_{dx}−HRV_{z})/(HRVₘₐₓ−HRV_{z}) if HRV_{dx}>HRV_{z}

SI_{HRV}=−(HRV_{dx}−HRV_{z})/(HRV_{z}−HRVₘᵢₙ) if HRV_{dx}<HRV_{z},

wherein a was selected as 0.25 and b as 0.5 and c and d were selected as 1. In the exemplary embodiment the current values of the stress index SI are selected after the predetermined number of pulse beats by means of a digital low-pass filter SI=f*SIₓ+(1−f)*SIₓ₋₁ calculated with f of 0.1. The device has been set up accordingly.

It should be still noted that the individual windows in which the stress index values are determined advantageously include various states of the subjects, such as e.g., lying, standing, moving, in the sense of the Conconi test, etc.

Moreover, it should be noted that even if test intervals are very widely separated in time, the adoption of the last test interval leads to a better result or more quickly to a good result than starting out with tabulated values. On the other hand, it may of course be appropriate to revert using tabulated values if the status of the subject has significantly changed in a fundamental manner.

We claim:

1. A processing device for determining a physiological condition of a person, the processing device being configured:
to receive data of a pulse frequency and of a heart rate variability of the person acquired by a wearable acquisition device;
to process the data of the pulse frequency and of the heart rate variability in a plurality of processing windows, comprising:
normalizing the pulse frequency using values of the pulse frequency in any processing window;
determining a first partial index value SIP based on the normalized pulse frequency;
normalizing the heart rate variability using values of the heart rate variability in any processing window;
determining a second partial index value SIHRV based on the normalized heart rate variability; and
determining an index indicative of the physiological condition of the person as a weighted function of the first partial index value SIP and the second partial index value SIHRV.

2. The processing device according to claim 1, wherein the processing device is further configured for controlling a display device for generating a visual representation of at least part of the indexes determined in the plurality of processing windows.

3. The processing device according to claim 1, wherein the indexes indicative of the physiological condition of the person are stress indexes between a high stress condition and a relaxed stress condition relative to the person's stress condition in any processing window and wherein a stress condition is detected and reported as the physiological condition.

4. The processing device according to claim 1, wherein the processing device is further configured to compare the indexes indicative of the physiological condition of the person with an alarm value indicative of an occurrence a physiological condition.

5. The processing device according to claim 1, wherein the plurality of processing windows are defined as a predetermined time interval and/or a predetermined number of heart beats.

6. The processing device according to claim 1, wherein:
the values of pulse frequency used for normalizing the pulse frequency comprise minimum and/or maximum values of the pulse frequency in any processing window, and/or
the values of heart rate variability used for normalizing the heart rate variability comprise minimum and/or maximum values of the heart rate variability in any processing window.

7. A system for determining a physiological condition of a person comprising:
a wearable acquisition device, such as a wearable electrocardiography device, for acquiring data of a pulse frequency and of a heart rate variability of the person;
a processing device according to claim 1, communicatively connected to the wearable acquisition device.

8. The system according to claim 7, further comprising a comparator device configured to compare the indexes indicative of the physiological condition of the person with an alarm value indicative of an occurrence a physiological condition.

9. The system according to claim 7, further comprising a display device for displaying a visual representation of at least part of the indexes determined in the plurality of processing windows.

10. A computer implemented method for determining a physiological condition of a person, the method comprising:
receiving data of a pulse frequency and of a heart rate variability of the person;
processing the data of the pulse frequency and of the heart rate variability in a plurality of processing windows, comprising:
normalizing the pulse frequency using values of the pulse frequency in any processing window;
determining a first partial index value SIP based on the normalized pulse frequency;
normalizing the heart rate variability using values of the heart rate variability in any processing window;
determining a second partial index value SIHRV based on the normalized heart rate variability; and
determining an index indicative of the physiological condition of the person as a weighted function of the first partial index value SIP and of the second partial index value SIHRV.

11. The method according to claim 10, wherein further comprising controlling a display device for generating a visual representation of at least part of the indexes determined in the plurality of processing windows.

12. The method according to claim 10, wherein the indexes indicative of the physiological condition of the person are stress indexes between a high stress condition and a relaxed stress condition relative to the person's stress condition in any processing window and wherein a stress condition is detected and reported as the physiological condition.

13. The method device according to claim 10, wherein further comprising comparing the indexes indicative of the physiological condition of the person with an alarm value indicative of an occurrence a physiological condition.

14. The method according to claim 10, wherein the plurality of processing windows are defined as a predetermined time interval and/or a predetermined number of heart beats.

15. The method according to claim 10, comprising:
using minimum and/or maximum values of the pulse frequency in any processing window for normalizing the pulse frequency comprise, and/or
using minimum and/or maximum values of the heart rate variability in any processing window for normalizing the heart rate variability.

16. Computer program product comprising instructions, which, when executed by a processing device, cause the processing device to carry out the method according to claim 10.
